SEQUENCE LISTING (5 sequences)

SEQ ID NO: 1 - protein, 34 aa, Lactobacillus plantarum
SVPTSVYTLGIKILWSAYKHRKTIEKSFNKGFYH

SEQ ID NO: 2 - protein, 29 aa, Lactobacillus plantarum
DLTTKLWSSWGYYLGKKARWNLKHPYVQF

SEQ ID NO: 3 - protein, 34 aa, Synthetic construct
LKLWNTYGTFSRFYTSKSEVKIAHGIKSIHVPYK

SEQ ID NO: 4 - protein, 29 aa, Synthetic construct
TWLKYGHGDAKLWSWSKPLNLTFRYQYRK

SEQ ID NO: 5 - protein, 37 aa, Homo sapiens
LLGDFFRKSKEKIGKEFKRIVQRIKDFLRNLVPRTES

(19) United States
(12) Patent Application Publication
(10) Pub. No.: US 2024/0374680 A1
(43) Pub. Date: Nov. 14, 2024

(54) PHARMACEUTICAL COMPOSITION FOR TREATMENT OF VIRAL INFECTIONS

(71) Applicant: Virenc AB, ÖREBRO (SE)

(72) Inventors: Hazem KHALAF, ÖREBRO (SE); Torbjörn BENGTSSON, LINKÖPING (SE); Jorma HINKULA, ÅTVIDABERG (SE); Wessam MELIK, LINDOME (SE); Robert SELEGÅRD, NORSHOLM (SE); Daniel AILI, LINKÖPING (SE)

(21) Appl. No.: 18/749,113

(22) Filed: Jun. 20, 2024

Related U.S. Application Data
(63) Continuation of application No. PCT/SE2022/051208, filed on Dec. 20, 2022.

(30) Foreign Application Priority Data
Dec. 21, 2021 (SE) 2151572-1

(51) Int. Cl. A61K 38/16; A61P 31/14; A61P 31/16; A61P 31/18

(52) U.S. Cl. CPC A61K 38/164; A61P 31/14; A61P 31/16; A61P 31/18

(57) ABSTRACT

The present disclosure is directed to a pharmaceutical composition for use in the prevention and/or treatment of a viral infection and/or a disorder associated with a viral infection, caused by an enveloped virus, wherein the pharmaceutical composition comprises: a) a peptide having at least 90% sequence identity to an amino acid sequence according to SEQ ID NO:1, which represents the peptide PLNC8 β; and/or b) a peptide having at least 90% sequence identity to an amino acid sequence according to SEQ ID NO:2, which represents the peptide PLNC8 α.

### CROSS-REFERENCE TO RELATED APPLICATIONS

This application is a continuation under 35 U.S.C. § 120 of International Application No. PCT/SE2022/051208, filed Dec. 20, 2022, which claims priority to Swedish Patent Application No. 2151572-1, filed Dec. 21, 2021, under 35 U.S.C. § 119(a). Each of the above-referenced patent applications is incorporated by reference in its entirety.

### REFERENCE TO THE ELECTRONIC SEQUENCE LISTING

The contents of the electronic sequence listing (E3789_001T_WO ST26 Sequence listing.xml; Size: 5,312 bytes; and Date of Creation: Jun. 20, 2024) are herein incorporated by reference in its entirety.

### TECHNICAL FIELD OF THE DISCLOSURE

The present disclosure relates to the prevention or treatment of viral infections and is directed to pharmaceutical compositions for use in the prevention or treatment of viral infections and disorders associated with a viral infection, caused by an enveloped virus, wherein the pharmaceutical composition comprises a peptide having at least 90% sequence identity to the amino acid sequence of the peptide plantaricin NC8 (PLNC8) β, and/or a peptide having at least 90% sequence identity to the amino acid sequence of the peptide PLNC8 α.

### BACKGROUND

Humans are constantly exposed to viruses and the consequences of a virus infection depends on numerous factors, such as virus type and its virulence properties, route of infection, and the immunological and inflammatory status and response of the host. The outcome of an infection may be lethal when the host is immunocompromized¹. Only a few antiviral drugs exist and these are expensive for widespread use and due to the complexity of viral pathogenesis treatment of viral infections today often includes symptom-reduction by targeting the host's inflammatory responses^{2,3}. Emerging viral pathogens, including SARS-CoV-2, are rapidly increasing and underscores our limited therapeutic resources against viral infections. No drugs have so far been approved to specifically inhibit SARS-CoV-2 infection and thereby treat COVID-19, which has resulted during recent year in a complete paralysis of many countries due to lockdown, to limit the spread of the virus and manage the recovery of infected patients^{4,5}. Thus, drugs specific against SARS-CoV-2 are still lacking, however some candidates are under preclinical/clinical trials. The majority of the approved antiviral drugs are used for the treatment of human immunodeficiency virus (HIV) by primarily targeting the nucleoside and non-nucleoside reverse transcriptase, viral protease activity and viral entry^{6,7}. Development of novel antiviral compounds against large classes of viruses, as opposed to single-pathogen therapeutics, would be more advantageous by treating a wide range of viral infections^{8,9}. Furthermore, development of antiviral substances that directly destroy the virions, e.g., by amphipathic peptides^{8,10}, would ultimately increase the efficacy by rapidly eliminating the viruses and their subsequent pathogenesis, and thereby limiting their dissemination to other organs and spreading between individuals. This is particularly interesting considering that vaccine development is a costly and time-consuming process and must regularly be updated as the RNA virus can mutate and render the vaccine ineffective. In addition, secondary bacterial infections associated with virus infections have been highlighted as a serious threat to human health, and Staphylococcus aureus is identified to be one of the most common pathogens^{11,12}. Consequently, a potent broadspectrum antiviral drug is urgently needed to combat emerging virus pathogens, including respiratory viruses such as influenza and corona viruses.

Bacteriocins are small antimicrobial peptides produced by bacteria, including lactobacilli, that kill microbes usually by membrane disruption^{13,14}. Bacteriocins have become attractive candidates for therapeutic applications in traditional medicine against bacterial infections due to their high potency, broad spectrum antibacterial activity and beneficial effects on human tissues. Plantaricin NC8 (PLNC8) αβ is a two-peptide bacteriocin expressed by Lactobacillus plantarum strains. We have previously reported that PLNC8 αβ effectively inhibits and kills several bacterial pathogens, including Staphylococcus spp., and markedly enhances the effects of antibiotics¹⁵. PLNC8 αβ consists of amphipathic peptides with positive net charges that display high affinity for negatively charged pathogen membrane structures, including phosphatidylglycerol rich bacterial membranes. This interaction leads to a rapid disruption of bacterial membrane integrity, permeabilization, and loss of homeostasis that eventually kills the bacteria. The anionic phospholipids in eukaryotic cell membranes are oriented toward the cytoplasmic leaflet, which is an active process that is regulated by a group of enzymes termed flippases (ATPases). Moreover, the high cholesterol content (˜40%) contributes to membrane stability and fluidity, and prevents membrane permeabilization by PLNC8 αβ and other similar peptides. Indeed, we have previously shown that PLNC8 αβ displays no cytotoxicity towards human cells, but rather induces cell proliferation and expression of several growth factors, including TGF-β1, IGF-1 and EGF¹⁶.

Viruses are broadly divided into two classes: enveloped and non-enveloped viruses. Both classes use host cells for their replication. Virus entry into host cells involves membrane fusion of the viral envelope with the plasma membrane or via receptor-mediated endosytosis. Enveloped viruses, such as influenzaviruses, coronaviruses, filoviruses and flaviviruses cover their protein capsid with a lipid envelope through budding from host cell membranes. For many virus families, e.g. coronaviridae and flaviridae, translation, replication, assembly and budding occur specifically in the endoplasmatic reticulum (ER), with following processing in the Golgi apparatus to produce mature viruses, which are then released through exocytosis. The lipid envelope of these viruses is thus derived from the ER. The lipid composition of the ER membrane differs from that of the plasma membrane, e.g., by an equal distribution of anionic lipids (for example phosphatidylserine) in both leaflets of the ER membrane, while the plasma membrane, in which all the charged lipids are oriented towards the cytosol, is asymmetric. The ER membrane however still contains cholesterol (˜8%) that provides stability and resistance against membrane-active amphipathic peptides^{17,18}.

The current pandemic by SARS-CoV-2 causes the life-threatening condition know as COVID-19, and vaccines have been developed through joint efforts. It still remains to be determined to what extent this virus is mutating into more virulent strains and if the newly developed vaccines are effective against these variants. While clarification of viral pathogenesis is an important step towards development of new therapeutics, effective broad range antiviral compounds are urgently needed considering the rapid emergence of new viral pathogens. Severe illness in patients with respiratory viral infections is often associated with underlying medical problems, such as cancer, diabetes, and cardiovascular disease. Infected patients must withstand severe suffering and longer hospital stay with special medical attention that in turn requires further medication and care, ultimately resulting in markedly increased health care costs. These challenges require global innovative actions considering that vaccine development is associated with high costs and restricted usage due to that virus, e.g., influenza, continuously evolve and mutate, causing the annual seasonal epidemics. One such strategy is to develop new broad-spectrum antivirals based on cationic peptides, that act on common elements shared by many viruses, to combat serious infections.

### SUMMARY OF THE DISCLOSURE

The object of the present disclosure is to provide a novel broadspectrum antiviral pharmaceutical composition. Herein, it is shown that the plantaricin NC8 (PLNC8) αβ, as well as its subunits, i.e., the two peptides PLNC8 α and PLNC8 β, exert antiviral activity and possess promising properties as antiviral agents against various viruses belonging to different virus families.

More particularly, the present disclosure is directed to a pharmaceutical composition for use in the prevention and/or treatment of a viral infection and/or a disorder associated with a viral infection, caused by an enveloped virus, wherein the pharmaceutical composition comprises: a) a peptide having at least 90%, such as 95%, 96%, 97%, 98%, or 99%, sequence identity to an amino acid sequence according to SEQ ID NO:1 (i.e., PLNC8 β); and/or b) a peptide having at least 90%, such as 95%, 96%, 97%, 98%, or 99%, sequence identity to an amino acid sequence according to SEQ ID NO:2 (i.e., PLNC8 α).

Preferred aspects of the present disclosure are described below in the detailed description and in the dependent claims.

### BRIEF DESCRIPTION OF THE DRAWINGS

FIG. 1 schematically illustrates the previously known composition and structure of lipids in the human plasma membrane and endoplasmic reticulum (ER) membrane.

FIG. 2A shows Cryo-Electron Microscopy (Cryo-EM) images of liposomes containing 5% phosphatidylserine, either with or without cholesterol, either left untreated (control) or treated with the peptide L-PLNC8 αβ. FIG. 2B shows the effect of the peptides L-PLNC8 α, L-PLNC8 β and L-PLNC8 αβ on liposomes containing 70% POPC and 30% cholesterol. FIG. 2C shows the effect of the peptides L-PLNC8 α, L-PLNC8 β and L-PLNC8 αβ on liposomes containing 91% POPC, 1% POPS and 8% cholesterol. FIG. 2D shows the effect of the peptides L-PLNC8 α, L-PLNC8 β and L-PLNC8 αβ on liposomes containing 81% POPC, 11% POPS and 8% cholesterol.

FIG. 3A shows microscopy images (Sytox Green fluorescence) of the effects on flavivirus LGTV after exposure to the peptides L-PLNC8 αβ, scrambled S-PLNC8 αβ, and LL-37, and untreated LGTV (Control). FIG. 3B is a graph of quantified LGTV plaques (focus-forming units, FFU) after exposure of the peptides at different concentrations, and untreated LGTV (Control). Representative images of the virus plaques are presented above the bars.

FIG. 4A shows images of the effects on LGTV after exposure to the peptides L-PLNC8 α and L-PLNC8 β, and untreated LGTV (Control). FIG. 4B is a graph of quantified LGTV plaques after exposure of the peptides, and untreated LGTV (Control). Representative images of the virus plaques are presented above the bars.

FIG. 5A shows images of the effects on LGTV after exposure to D-PLNC8 α, D-PLNC8 β and D-PLNC8 αβ, and untreated LGTV (Control). FIG. 5B is a graph of quantified LGTV plaques after exposure of the peptides, and untreated LGTV (Control). Representative images of the virus plaques are presented above the bars.

FIG. 6 shows Transmission Electron Microscopy (TEM) images visualizing LGTV particles within cells.

FIG. 7A is a graph of quantified flavivirus KUNV plaques after exposure to L-PLNC8 αβ, L-PLNC8 α and L-PLNC8 β. FIG. 7B is a graph of quantified KUNV plaques after exposure to D-PLNC8 αβ, D-PLNC8 α and D-PLNC8 β.

FIG. 8 is a graph showing the antiviral activity of L- and D-enantiomers of PLNC8 αβ against different virus titers of KUNV.

FIG. 9A-B are graphs showing the effects of L- and D-enantiomers of PLNC8 αβ on KUNV virus replication (FIG. 9A), and accumulation of extracellular virions (FIG. 9B).

FIG. 10A-B are graphs showing the antiviral activity of the L-enantiomer of PLNC8 αβ and PLNC8 β (FIG. 10A) and the D-enantiomer of PLNC8 αβ and PLNC8 β (FIG. 10B) against SARS-CoV-2.

FIG. 11A-B are graphs showing the antiviral activity of the L-enantiomer of PLNC8 αβ and PLNC8 β (FIG. 11A) and the D-enantiomer of PLNC8 αβ and PLNC8 β (FIG. 11B) against Influenza A virus.

FIG. 12A-B are graphs showing the effect of the L-enantiomer of PLNC8 αβ and PLNC8 β (FIG. 12A) and the D-enantiomer of PLNC8 αβ and PLNC8 β (FIG. 12B) against HIV-1.

FIG. 13A-B are graphs showing the effect of the L-enantiomer of PLNC8 αβ and the D-enantiomer of PLNC8 αβ, respectively, on morphology (FIG. 13A) and viability (FIG. 13B) of non-infected cells or KUNV-infected cells.

FIG. 14 is a graph showing the effects of L- and D-enantiomers of PLNC8 αβ on human lung carcinoma cells infected with KUNV.

### DETAILED DESCRIPTION OF THE DISCLOSURE

The present disclosure solves or at least mitigates the above-described problems associated with currently available preventative and treatment measures against viral infections, by providing a pharmaceutical composition for use in the prevention and/or treatment of a viral infection and/or a disorder associated with a viral infection, caused by an enveloped virus, wherein the pharmaceutical composition comprises: a) a peptide having at least 90%, such as 95%, 96%, 97%, 98%, or 99%, sequence identity to an amino acid sequence according to SEQ ID NO:1 (i.e., PLNC8 β); and/or b) a peptide having at least 90%, such as 95%, 96%, 97%, 98%, or 99%, sequence identity to an amino acid sequence according to SEQ ID NO:2 (i.e., PLNC8 α).

Significant advantages of the presently disclosed pharmaceutical composition include that the peptides comprised therein have been shown to have potent activity against enveloped viruses through permeabilizing actions against the viral envelope. More particularly, it is shown in the experimental section herein that permeabilization is effected by the peptides' activity through electrostatic interactions with anionic lipids of the envelope, and thus specifically targeting extracellular virions without affecting human cell viability. This hypothesis has been tested and verified by experimental micobiological studies investigating the effects of PLNC8 αβ against different viral species, in combination with previous knowledge of the composition of different biological membranes.

Further, it is shown herein that the concentration of PLNC8 αβ that is required to eliminate all the infective virus particles is in the range of nanomolar (nM) to micromolar (μM), which is surprisingly efficient considering the high content of cholesterol (8-35%) in their lipid envelopes. PLNC8 αβ can thus be used as an effective antiviral agent, independent of virus antigenic mutations as it targets the virus envelope structure. This structure is stable and not dependent on the coding genome of the virus but is derived from infected host cells, including the plasma membrane, endoplasmic reticulum, Golgi complex, and nuclear envelope. Consequently, the differences in structure and function between the viral envelope and host cell plasma membrane make viral membranes ideal targets for antiviral therapy by using membrane-active amphipathic peptides such as PLNC8 αβ.

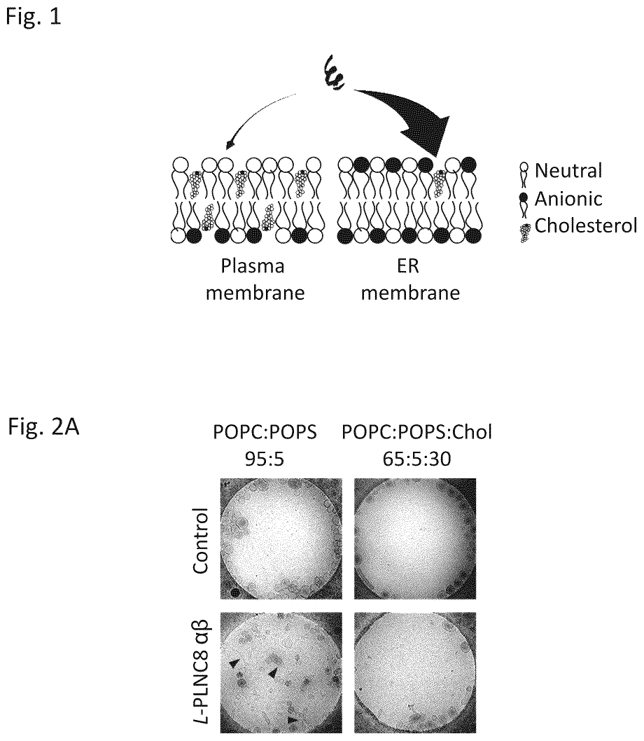

Previous results showing that PLNC8 αβ displays no cytotoxicity towards human cells^{15,16} suggested that, since enveloped viruses replicate and bud from human cells, it is unlikely that PLNC8 αβ possesses any antiviral properties. More particularly, since eukaryotic membranes contain cholesterol and have a specific phospholipid composition and orientation, it is unlikely that amphipathic peptides, including PLNC8 αβ, would have any antiviral properties against enveloped viruses since these viruses exploit the translational machinery intracellularly and bud off from the plasma membrane of human cells. Nevertheless, the present inventors hypothesized that PLNC8 αβ would in fact have antiviral activity. This hypothesis is based on the apparent differences in cholesterol content, phospholipid composition, and anionic charge of the outer leaflet between the plasma membrane and cell organelles, e.g., the ER membrane. FIG. 1 illustrates that the plasma membrane has higher cholesterol content (35%) than the ER membrane (8%), and furthermore, that the plasma membrane is asymmetric, in which the extracellular leaflet is composed of zwitterionic phospholipids, and all anionic phospholipids are oriented towards the cytosol, while the ER membrane is symmetric where both leaflets contain anionic phospholipids. In the experimental section herein, it is also shown that the differences between the plasma membrane and the ER membrane are crucial determining factors enabling PLNC8 αβ to efficiently permeabilize viral envelopes without affecting cell viability.

The viral families studied herein have distinctly different replication pathways. They replicate and assemble in different cell types and intracellular localities, which may explain the variable sensitivity to the antiviral peptides that have been tested herein. Indeed, it was found that viruses that use the ER/Golgi complex pathway, e.g., SARS-CoV-2 and flaviviruses, are considerably more susceptible to PLNC8 αβ, compared to viruses that acquire their lipid envelope from the plasma membrane, such as Influenza A virus and HIV-1. Nevertheless, the results show that the peptides tested exert an antiviral effect on distinctly different enveloped viruses belonging to a number of different viral families. Further, as shown herein, the antiviral effect is achieved by permeabilization of the viral envelope and is not dependent on the coding genome of the virus. Consequently, the peptides may exert an antiviral effect on any type of enveloped viruses.

As mentioned above, the present disclosure is directed to a pharmaceutical composition for use in the prevention and/or treatment of a viral infection and/or a disorder associated with a viral infection, caused by an enveloped virus, wherein the pharmaceutical composition comprises a peptide having at least 90% sequence identity to an amino acid sequence according to SEQ ID NO:1 or SEQ ID NO: 2, respectively. It is to be understood that such a peptide having at least 90% sequence identity must also be functionally equivalent to the amino acid sequence according to SEQ ID NO: 1 or 2, respectively, in terms of its antiviral effect or activity. The antiviral effect or activity of a peptide may be determined by performing any one or more of the experiments described in the experimental section below.

Also described herein is a peptide for use in the prevention and/or treatment of a viral infection and/or a disorder associated with a viral infection, caused by an enveloped virus, wherein the peptide is a) a peptide having at least 90%, such as 95%, 96%, 97%, 98%, or 99%, sequence identity to an amino acid sequence according to SEQ ID NO:1;
or b) a peptide having at least 90%, such as 95%, 96%, 97%, 98%, or 99%, sequence identity to an amino acid sequence according to SEQ ID NO:2. As above, it is to be understood that such a peptide having at least 90% sequence identity must also be functionally equivalent to the amino acid sequence according to SEQ ID NO: 1 or 2, respectively, in terms of its antiviral effect or activity. Again, the antiviral effect or activity of a peptide may be determined by performing any one or more of the experiments described in the experimental section below.

Further, it is to be understood that the above-mentioned peptide(s) may be formulated in any pharmaceutically acceptable manner. For example, the peptide(s) may be in the form of a pharmaceutically acceptable salt or solvate.

The above-mentioned peptide(s), originally composed of L-amino acids, may comprise at least one D-amino acid residue, such as two, three, four etc. D-amino acid residues, up to and including the total amount of amino acid residues present in the peptide(s).

Thus, in the herein described pharmaceutical composition, at least one amino acid residue of the peptide according to (a) and/or (b) (as defined above) may be a D-amino acid residue, such as two, three, four etc. D-amino acid residues, up to and including the total number of amino acid residues of the peptide according to (a) and/or (b) (as defined above). Alternatively, in the pharmaceutical composition at least about 3%, such as about 3.5%, about 5%, about 10%, about 20%, about 30%, about 40%, about 50%, about 60%, about 70%, about 80%, about 90%, or about 100%, of the amino acid residues of the peptide according to (a) and/or (b) (as defined above) may be D-amino acid residues.

The herein disclosed pharmaceutical composition for use in the prevention and/or treatment of a viral infection and/or a disorder associated with a viral infection, caused by an enveloped virus, may comprise from about 1 nM to about 1000 μM, such as from about 10 nM to about 100 μM, or such as about 1 nM, 10 nM, 50 nM, 100 nM, 500 nM, 1 μM, 10 μM, 50 μM, 100 μM, 500 μM, or 1000 μM, of the peptide according to (a) and/or (b).

The herein disclosed pharmaceutical composition for use in the prevention and/or treatment of a viral infection and/or a disorder associated with a viral infection, caused by an enveloped virus, may comprise the peptides according to (a) and (b) at a molar ratio of from about 1:1 to about 20:1, such as from about 1:1 to about 10:1, such as about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1.

In the context of the herein disclosed pharmaceutical composition for use in the prevention and/or treatment of a viral infection and/or a disorder associated with a viral infection, the viral infection may be caused by an enveloped virus selected from the group consisting of the following virus families: Coronaviridae, Flaviviridae, Herpesviridae, Orthomyxoviridae, Retroviridae, Paramyxoviridae, Filoviridae, Pneumoviridae, Arteriviridae, Asfarviridae, Bunyaviridae, Hepadnaviridae, Poxviridae, Togaviridae and Rhabdoviridae.

Accordingly, as a non-limiting example, the enveloped virus may belong to Coronaviridae.

Alternatively, as a non-limiting example, the enveloped virus may belong to Flaviviridae.

Alternatively, as a non-limiting example, the enveloped virus may belong to Herpesviridae.

Alternatively, as a non-limiting example, the enveloped virus may belong to Orthomyxoviridae.

Alternatively, as a non-limiting example, the enveloped virus may belong to Retroviridae.

Alternatively, as a non-limiting example, the enveloped virus may belong to Paramyxoviridae.

Alternatively, as a non-limiting example, the enveloped virus may belong to Filoviridae.

Alternatively, as a non-limiting example, the enveloped virus may belong to Pneumoviridae.

Alternatively, as a non-limiting example, the enveloped virus may belong to Arteriviridae.

Alternatively, as a non-limiting example, the enveloped virus may belong to Asfarviridae.

Alternatively, as a non-limiting example, the enveloped virus may belong to Bunyaviridae.

Alternatively, as a non-limiting example, the enveloped virus may belong to Hepadnaviridae.

Alternatively, as a non-limiting example, the enveloped virus may belong to Poxviridae.

Alternatively, as a non-limiting example, the enveloped virus may belong to Togaviridae.

Alternatively, as a non-limiting example, the enveloped virus may belong to Rhabdoviridae.

Alternatively, the viral infection may be caused by an enveloped virus, whose envelope is obtained from the endoplasmic reticulum, the golgi apparatus or the nuclear envelope of the infected cell, such as an enveloped virus selected from the group consisting of the following virus families: Coronaviridae, Flaviviridae, Herpesviridae, Arteriviridae, Asfarviridae, Bunyaviridae, Hepadnaviridae and Poxviridae.

Accordingly, as a non-limiting example, the enveloped virus, whose envelope is obtained from the endoplasmic reticulum, the golgi apparatus or the nuclear envelope of the infected cell, may belong to Coronaviridae.

Alternatively, as a non-limiting example, such an enveloped virus may belong to Flaviviridae.

Alternatively, as a non-limiting example, such an enveloped virus may belong to Herpesviridae.

Alternatively, as a non-limiting example, such an enveloped virus may belong to Arteriviridae.

Alternatively, as a non-limiting example, such an enveloped virus may belong to Asfarviridae.

Alternatively, as a non-limiting example, such an enveloped virus may belong to Bunyaviridae.

Alternatively, as a non-limiting example, such an enveloped virus may belong to Hepadnaviridae.

Alternatively, as a non-limiting example, such an enveloped virus may belong to Poxviridae.

Non-limiting examples of such enveloped viruses are Severe Acute Respiratory Syndrome Coronavirus (SARS-CoV), Severe Acute Respiratory Coronavirus 2 (SARS-CoV-2), Middle East Respiratory Syndrome Coronavirus (MERS-CoV), Herpes Simplex Virus 1 (HSV-1), Herpes Simplex Virus 2 (HSV-2), Epstein-Barr Virus (EBV), Human Cytomegalovirus (HCMV), Varicella-Zoster Virus (VZV), Dengue Virus (DENV), Zika Virus (ZIKV), West Nile Virus (WNV), Langat Virus (LGTV) and Tick-borne Encephalitis Virus (TBEV).

Accordingly, as a non-limiting example, such an enveloped virus may be Severe Acute Respiratory Syndrome Coronavirus (SARS-CoV).

Alternatively, as a non-limiting example, such an enveloped virus may be Severe Acute Respiratory Coronavirus 2 (SARS-CoV-2).

Alternatively, as a non-limiting example, such an enveloped virus may be Middle East Respiratory Syndrome Coronavirus (MERS-CoV).

Alternatively, as a non-limiting example, such an enveloped virus may be Herpes Simplex Virus 1 (HSV-1).

Alternatively, as a non-limiting example, such an enveloped virus may be Herpes Simplex Virus 2 (HSV-2).

Alternatively, as a non-limiting example, such an enveloped virus may be Epstein-Barr Virus (EBV).

Alternatively, as a non-limiting example, such an enveloped virus may be Human Cytomegalovirus (HCMV).

Alternatively, as a non-limiting example, such an enveloped virus may be Varicella-Zoster Virus (VZV).

Alternatively, as a non-limiting example, such an enveloped virus may be Dengue Virus (DENV).

Alternatively, as a non-limiting example, such an enveloped virus may be Zika Virus (ZIKV).

Alternatively, as a non-limiting example, such an enveloped virus may be West Nile Virus (WNV).

Alternatively, as a non-limiting example, such an enveloped virus may be Langat Virus (LGTV).

Alternatively, as a non-limiting example, such an enveloped virus may be Tick-borne Encephalitis Virus (TBEV).

Another non-limiting example of such an enveloped virus is Kunjin virus (KUNV), which is a subtype of the above-mentioned West Nile Virus (WNV).

The viral infection to be prevented and/or treated by use of the herein described pharmaceutical composition may be a respiratory tract infection (such as an upper respiratory tract infection or a lower respiratory tract infection) and/or a mucus layer infection.

A non-limiting example of an enveloped virus which may cause a respiratory tract infection and/or a mucus layer infection is Severe Acute Respiratory Syndrome Coronavirus (SARS-CoV).

Another non-limiting example of an enveloped virus which may cause a respiratory tract infection and/or a mucus layer infection is Severe Acute Respiratory Coronavirus 2 (SARS-CoV-2).

Another non-limiting example of an enveloped virus which may cause a respiratory tract infection and/or a mucus layer infection is Middle East Respiratory Syndrome Coronavirus (MERS-CoV).

Another non-limiting example of an enveloped virus which may cause a respiratory tract infection and/or a mucus layer infection is Herpes Simplex Virus 1 (HSV-1).

Another non-limiting example of an enveloped virus which may cause a respiratory tract infection and/or a mucus layer infection is Herpes Simplex Virus 2 (HSV-2).

Another non-limiting example of an enveloped virus which may cause a respiratory tract infection and/or a mucus layer infection is Epstein-Barr Virus (EBV).

Another non-limiting example of an enveloped virus which may cause a respiratory tract infection and/or a mucus layer infection is Human Cytomegalovirus (HCMV).

Another non-limiting example of an enveloped virus which may cause a respiratory tract infection and/or a mucus layer infection is Varicella-Zoster Virus (VZV).

Throughout this text, the expression “disorder associated with a viral infection” is intended to mean a disorder that is induced by, or caused by, a viral infection. In other words, the disorder may alternatively be said to be a “virus-induced disorder”.

The term “disorder” may alternatively be described as a “disease”, “medical complication”, “physical complication”, or, in short, “complication”, and these terms may be used interchangeably herein.

The peptides and pharmaceutical compositions described herein are shown to exert a direct antiviral effect on enveloped viruses. Hence, the peptides and pharmaceutical compositions will exert a preventative or therapeutic effect against any disorder induced by an enveloped virus. Thanks to their direct antiviral effects, it is possible to prevent or treat a disorder associated with a viral infection irrespective of whether the disorder occurs early or late in the chain of events induced by a viral infection in a host organism, and irrespective of whether the disorder is caused directly by the physical interactions between a virus and its host cells or is caused by another disorder which in turn has been caused by an underlying viral infection.

Herein, non-limiting examples of a disorder associated with a viral infection are virus-induced inflammation, virus-induced cell death, virus-induced tissue destruction, and combinations thereof. Further, non-limiting examples of virus-induced tissue destruction are damage of mucosal surfaces, pulmonary fibrosis, organ dysfunction, and combinations thereof. More particularly, non-limiting examples of disorders associated with infections by respiratory viruses (e.g., Influenza A and SARS-CoV-2) are cell death (caused by budding of virus particles), tissue destruction, fever, and cold symptoms, such as a sore throat, abundant snot production, congestion in the upper airways, and coughing, as well as pneumonia and pulmonary fibrosis. Non-limiting examples of disorders associated with an HIV infection are destruction of lymphocytes, and susceptibility to infections caused by pathogenic microorganisms. A non-limiting example of a disorder associated with a viral infection by for example a Herpes Simplex virus is viral sepsis.

The pharmaceutical composition for use as disclosed herein may be administered locally to the site of infection, or close to the site of infection. A person skilled in the art (such as a medical practitioner) readily understands how close to a site of infection it is relevant and/or necessary to administer the pharmaceutical composition to achieve the desired medical effects of the composition. The pharmaceutical composition for use as disclosed herein may for example be administered topically, locally to the site of infection or close to the site of infection.

The pharmaceutical composition for use as disclosed herein may be formulated as a powder, a solution, a cream, a gel, an ointment, or is formulated in immobilized form as a coating on a medical device.

Where the pharmaceutical composition is formulated as a powder, it may for example be administered via an inhaler (i.e., a portable device for administering a drug which is to be breathed in).

Where the pharmaceutical composition is formulated as a solution, the solution may optionally be an aerosol and/or in the form of a nasal spray, mouth spray, or administered via an inhaler.

Where the pharmaceutical composition is formulated in immobilized form as a coating on a medical device, the medical device may optionally be a face mask, an air filter, a nasal cannula device or an endotracheal tube.

The present disclosure is also useful for a medical device which is at least partly coated with a pharmaceutical composition comprising a peptide according to (a) and/or (b) as defined above.

The pharmaceutical composition for use as disclosed herein may, in addition to the above-described peptide(s), comprise one or more pharmaceutically acceptable excipient(s). Non-limiting examples of such an excipient is a solubilizer, a surfactant, a bulking agent, a thickener, a preservative, a vehicle, a salt, a sugar, and a buffering agent.

The pharmaceutical composition for use as disclosed herein may be formulated for administration as a single dose or multiple doses, such as two, three, four, or five doses per day, for 1-20 days, such as 3-20 days, or 1, 2, 3, 4, 5, 6, 7, 8, 9, 10, 11, 12, 13, 14, 15, 16, 17, 18, 19, or 20 days.

The pharmaceutical composition for use as disclosed herein may, in addition to the above-described peptide(s) and optionally one or more pharmaceutically acceptable excipient(s) as mentioned above, comprise one or more additional antiviral agent(s).

Non-limiting examples of antiviral agents are an antiviral agent intended for use in the prevention or treatment of an infection caused by Herpes simplex, such as Acyclovir or a functional equivalent thereof, and/or an antiviral agent intended for use in the prevention or treatment of infections caused by Influenza A or B, such as Oseltamivir (Tamiflu) or a functional equivalent thereof, and/or an antiviral agent intended for use in the prevention or treatment of an infection caused by SARS-CoV-2, such as Remdesivir or a functional equivalent thereof.

The present disclosure is also useful for a kit of parts comprising:
(i) a pharmaceutical formulation including an antiviral agent, optionally in admixture with a pharmaceutically acceptable excipient; and
(ii) a peptide according to (a) and/or (b) as defined further above, or a pharmaceutical composition comprising a peptide according to (a) and/or (b) as defined further above.

The present disclosure is further useful in the context of an antiviral agent for use in a method for the prevention and/or treatment of a viral infection and/or a disorder associated with a viral infection, as defined elsewhere herein, wherein said use comprises administration of said antiviral agent in combination with a pharmaceutical composition comprising a peptide according to (a) and/or (b) as defined further above.

The present disclosure is additionally useful for a method for the prevention and/or treatment of a viral infection and/or a disorder associated with a viral infection, wherein said method comprises administering a therapeutically effective amount of a pharmaceutical composition comprising a peptide according to (a) and/or (b) as defined further above, to a subject in need thereof.

The present disclosure is also useful for a method for the prevention and/or treatment of a viral infection and/or a disorder associated with a viral infection, wherein said method comprises administering a therapeutically effective amount of an antiviral agent and a pharmaceutical composition comprising a peptide according to (a) and/or (b) as defined further above, to a subject in need thereof. Optionally, said antiviral agent and said pharmaceutical composition may be present in the same pharmaceutical formulation.

### Experimental Section

### Materials and Methods

### Cell Culture

Monkey (Cercopithecus aethiops) epithelial kidney cells (Vero E6, ATCC, CCL-81), dog (Canis familiaris) epithelial kidney cells (MDCK, ATCC, NBL-2), and human lung carcinoma cells (A549, ATCC, CCL-185) were maintained in Dulbecco's Modified Eagle's Medium (DMEM) containing 1 g/L glucose (Gibco), supplemented with 10% heat-inactivated fetal bovine serum (HI-FBS, Gibco) and 100 U/mL penicillin-streptomycin (PEST, Gibco) at 37° C. in 5% CO₂. Jurkat T-cells (E6-1, ATCC) were maintained in RPMI 1640 medium (Fisher scientific, Austria) with 1.5 mM L-glutamine (Invitrogen, USA) and supplemented with 10% FBS. The cells were incubated in a stable environment at 95% air, 5% CO₂ and 37° C. Peripheral blood mononuclear cells (PBMC) were isolated by the density gradient medium Ficoll-Paque™ Plus (Amersham Biosciences, Sweden) according to the manufacturers' instructions. Briefly, freshly collected blood from healthy donors was diluted with an equal volume of PBS, and 4 ml were carefully layered on top of 3 ml Ficoll-Paque Plus, whereafter the tubes were centrifuged at room temperature for 30 min at 300×g. PBMC were recovered from the interface and washed twice with PBS to remove excess Ficoll-Paque Plus and platelets. The cells were suspended in RPMI media supplemented with 10% FBS and maintained at 95% air, 5% CO₂ and 37° C. for subsequent experiments.

### Virus Strains and Propagation

Virus propagation was initiated by infecting Vero cells with Langat virus (LGTV), West Nile virus (WNV), Kunjin virus (KUNV), Human Immunodeficiency virus (HIV)-1 (subtype B, MN strain), Influenza A virus (H1N1/CA09pdm), or Severe Acute Respiratory Syndrome Coronavirus 2 (β-SARS-CoV-2) at a multiplicity of infection (MOI) of 0.1. Infected cells were then grown in complete DMEM at 37° C. in 5% CO₂ until a cytopathic effect (CPE) was observed at 4-6 days post-infection. Following the observation of a CPE, the cell culture medium was semi-purified by ultracentrifugation over a 20% sucrose cushion at 150,000×g and 4° C. for 2.5 h. The virus-containing pellet was resuspended in complete DMEM medium, and the virus concentration was quantified by performing plaque assays.

### Peptides

The peptides used in the experiments herein were purchased from GL Biochem (Shanghai) Ltd, China. Their names, amino acid sequences, molecular weights, and net charge at pH 7, as well as their SEQ ID NO:s, are presented in Table 1 below. In the peptides denoted L-PLNC8 α and L-PLNC8 β¹³, all amino acid residues are in the L-configuration. In the peptides denoted D-PLNC8 α and D-PLNC8 β, all amino acid residues are in the D-configuration. Both L-PLNC8 β and D-PLNC8 β have an amino acid sequence according to SEQ ID NO:1, while both L-PLNC8 α and D-PLNC8 α have an amino acid sequence according to SEQ ID NO:2. Scrambled forms of PLNC8 α and PLNC8 β (denoted S-PLNC8 α and S-PLNC8 β¹⁵, respectively) were generated by randomly displacing the amino acids of the original sequences to show that the order of the amino acids is important for the activity of PLNC8 α and PLNC8 β. The human cathelicidin-derived peptide LL-37 is a known bactericidal peptide.

TABLE 1
Name, amino acid sequence, molecular weight,
net charge at pH 7 and SEQ ID NO of the
peptides used in the experiments herein.

| | | | Net | |
|---|---|---|---|---|
| | | | charge | SEQ |
| | | | at | ID |
| Name | Sequence | MW | pH 7 | NO |
| L-PLNC8 | DLTTKLWSSWGYYLGKKAR | 3587 | 4.1 | 2 |
| α | WNLKHPYVQF | | | |
| D-PLNC8 | DLTTKLWSSWGYYLGKKAR | 3587 | 4.1 | 2 |
| α | WNLKHPYVQF | | | |
| S-PLNC8 | TWLKYGHGDAKLWSWSKPL | 3587 | 4.1 | 4 |
| α | NLTFRYQYRK | | | |
| L-PLNC8 | SVPTSVYTLGIKILWSAYK | 4001 | 5.2 | 1 |
| β | HRKTIEKSFNKGFYH | | | |
| D-PLNC8 | SVPTSVYTLGIKILWSAYK | 4001 | 5.2 | 1 |
| β | HRKTIEKSFNKGFYH | | | |
| S-PLNC8 | LKLWNTYGTFSRFYTSKSE | 4001 | 5.2 | 3 |
| β | VKIAHGIKSIHVPYK | | | |
| LL-37 | LLGDFFRKSKEKIGKEFKR | 4493 | 6.0 | 5 |
| | IVQRIKDFLRNLVPRTES | | | |

### Liposome Preparation

Liposomes were prepared by thin film hydration, followed by extrusion¹⁹. Stock solutions of the lipids 1-palmitoyl-2-oleoyl-sn-glycero3-phosphatidylcholine (POPC), 1-palmitoyl-2-oleoyl-sn-glycero-3-phosphoL-serine (POPS) and cholesterol (Chol) were mixed at molar ratio 95:0:5, 65:30:5, 70:0:30, 91:1:8 and 81:11:8. The solvent was slowly evaporated via nitrogen stream, then the vials were dried in a vacuum desiccator overnight. Dry films were hydrated by addition of 1 mL 5(6)-carboxyfluorescein solution. The CF solution, presenting self-quenching concentration of CF, was prepared with 50 mM CF dissolved in 10 mM PB buffer and 90 mM NaCl, followed by pH adjustment. CF solution was dispensed on the lipid cake and incubated for 10 min under gentle shaking (50 min-1 on orbital shaker), followed by 1 min vortexing. The liposomes were extruded 21 times with a mini extruder (Avanti Polar Lipids, Inc.), through a 0.1 um membrane (Nucleapore track-etched hydrophilic membrane). The lipids were purified by gel filtration through a PD Mini Trap G-25 column by elution in PB buffer (10 mM).

### Carboxyfluorescein (CF) Release Assay

To study peptide efficacy on liposome membrane disruption, the liposome fluorescence was monitored upon peptide addition by microplate reader (Tecan Infinite M1000 Pro, λₑₓ=485 nm, λₑₘ=520 nm). Liposomes were diluted to a final concentration of 25 μM with 10 mM PB buffer and incubated with PLNC8 α, PLNC8 β and PLNC8 α/β at concentration of 10⁻⁵-10² μM in a 96 well plate at a final volume of 200 μL. Due to the self-quenching concentration of carboxyfluorescein (CF) inside the liposomes, any fluorescence of the liposome-peptide system could be attributed to the liposome membrane disruption. Fluorescence was monitored prior to peptide addition (F₀), and continuatively for 30 min. Total CF release (F_{T}) was achieved through addition of 1% Triton X-100. Instantaneous CF release (%) was calculated through the formula 100×(F−F0)/(FT−F0), where F is the instantaneous fluorescence.

### Flavivirus Inhibition Assay

The antiviral activity of PLNC8 αβ was determined using plaque assay. Crystal violet-based plaque assay was performed to quantify KUNV and immunofocus plaque assay was performed to quantify LGTV. Briefly, a series of virus dilutions, untreated or pre-exposed to PLNC8 αβ (forms and ratios as indicated in the examples below), in DMEM were used to infect a 90% confluent layer of Vero cells for 1 h at 37° C., followed by cell-overlaying with DMEM supplemented with 1.2% Avicel (FMC), 2% HI-FBS (Gibco), 1× non-essential amino acids (Gibco), and 1% PEST (Gibco). After 3-5 days, the overlays were removed, and cells were fixed by methanol for 20 min before proceeding with the plaque assays. For immunofocus assay, the fixed cells were blocked with 2% BSA (Fitzgerald) for 10 min before being labeled with mouse anti-E antibody (1:1000, anti-TBEV-E, United States Army Medical Research, Institute of Infectious Diseases, Fort Detrick, Frederick, MD, USA), followed by addition of anti-mouse secondary HRP Polymer (1:100) for 1 h at 37° C., and finally KPL TrueBlue Peroxidase Substrate (Seracare) for 15 min at room temperature (RT). For crystal violet-based plaque assay, the fixed cells were stained with 2% crystal violet (Sigma), 20% methanol (Fisher), and 0.1% ammonium oxalate (Sigma) solution. The number of virus plaques was quantified, and representative images of the wells were captured with Olympus SZX9 at 10× magnification and processed using the software ImageJ.

### SARS-CoV-2 Virus Inhibition Assay

The SARS-CoV-2 virus neutralization assay was used to quantify antiviral peptide activity against SARS-CoV-2 virus²⁰⁻²². In brief, the peptides (L-PLNC8 αβ and D-PLNC8 αβ, at a molar ratio of 1:1) were diluted in DMEM-0.1% FCS and PEST were incubated with the selected virus dose 100 Plaque Forming Units (PFU)/0.1 ml at 37° C. for 30 min, followed by transfer of the peptide-virus mixture to semi-confluent Vero E6 cells (85-95% confluency). Untreated virus-samples were also included. The peptide-virus suspension was kept with cells for 1 h before cells were washed and agarose gel (2%) was added followed by addition of 200 μl DMEM with 5% FCS on top of the gel. Cell cultures were kept at 37° C. for 72-96 h before the assay was terminated. Cells were rinsed with sterile PBS, before fixation for 45 min. The cells were stained with 2% crystal violet (Merck, Sigma-Aldrich, Sthlm, Sweden) for 45 min before the wells were emptied, washed twice with sterile PBS, and dried. Plaques were calculated in a plate microscope. A neutralizing titer endpoint dilution where no viral plaques (i.e., 0 PFU) were visible was defined as a protective amount of the antiviral peptides.

### Influenza A Virus Inhibition Assay

The influenza A virus neutralization assay²³ was used to quantify antiviral peptide activity against influenza A virus. In brief, DMEM supplemented with 0.1% FCS and PEST, with or without peptides, was inoculated with the selected virus dose of 100 PFU/0.1 ml and incubated at 37° C. for 30 min followed by transfer of the peptide-virus mixture and untreated virus-samples to semi-confluent MDCK cells. The peptide-virus suspension was kept with cells for 1 h before cells were washed, agarose gel (2%) was added and 200 μl DMEM with 5% FCS was put on-top of the gel. Cell cultures were kept at 37° C. for 72-96 h before assay was terminated. Cells were rinsed with sterile PBS, before fixation for 45 min. The cells were stained with 2% crystal violet (Merck, Sigma-Aldrich, Sthlm, Sweden) for 45 min before the wells were emptied, washed twice with sterile PBS, and dried. Plaques were calculated in a plate microscope. A neutralizing titer endpoint dilution where no viral plaques were visible (i.e., 0 PFU) was defined as a protective amount of the antiviral peptides.

### HIV-1 Neutralization Assay.

The HIV-1 neutralization was performed as previously described^{24,25}. In brief, viral isolates were derived from HIV-1 subtype B MN strain, subtype B strain (http://www.hiv.lanl.gov). Antiviral peptides were diluted in RPMI 1640 supplemented with 5% FCS and PEST in 96-well tissue culture plates (Costar, 260860, ThermoFisher Scientific). Each peptide concentration was mixed with virus at 50 tissue cell-culture infectious dose 50 (50TCID₅₀) and incubated at 37° C. for 1 h followed by the addition of 10⁵ human PBMCs/0.1 ml and activated by phytohemagglutinin (PHA) and rIL-2 (200-02, PeproTech) or (10⁵ c/0.1 ml) Jurkat T cells. The cells were incubated 120 min. at 37° C. in 5% CO₂ in air and washed twice with RPMI 1640 with 5% FCS before they received new medium. After 3 days, 50% of the medium was exchanged and at 5-7 days of culture, the presence of HIV-1 p24 antigen in the culture medium was measured by capture-ELISA for HIV-1 subtype B. The background of the p24 was determined for each plate and subtracted from all wells. The percentage of neutralization was determined as [1−(mean p24 OD in the presence of test medium/mean p24 OD in the absence of test serum)]×100.

### Cytotoxicity

Cytotoxicity was determined by assessing the activity of lactate dehydrogenase (LDH) in cell culture supernatants, using LDH Cytotoxicity Assay Kit, (Thermo Scientific™) and following the instructions of the manufacturer. The principle of the assay is based on that intracellular LDH is released by damaged cells, and ultimately changes the chemicals used in the assay to a colored formazan product that can be measured at 490 nm and its level is directly proportional to the amount of LDH released.

### Reverse Transcription PCR (RT-PCR)

RT-PCR was used to determine expression levels of several human genes and KUNV mRNA levels in response to KUNV infection, with or without L-PLNC8 αβ or D-PLNC8 αβ. Briefly, human lung carcinoma cells (A549) were infected with KUNV for 1 h, followed by treatment with 10 μM of L- or D-PLNC8 αβ for 48 h. RNA was extracted using RNeasy® Plus Micro Kit (Qiagen, USA) according to the manufacturer's recommendations. Reverse transcription was performed using Maxima® First Strand cDNA Synthesis Kit (Fermentas, Sweden). Thermal cycling conditions for SYBR Green (Fermentas) consisted of a denaturation step at 95° C. for 10 min followed by 40 cycles of 95° C. for 15 s and 60° C. for 60 s. Gene expression was analyzed using a 7900 HT real-time PCR instrument (Applied Biosystems). The obtained Ct values were normalized against GAPDH. Relative quantification of gene-expression was determined by using the ΔΔCt method. The ΔCt was calculated by subtracting the Ct of GAPDH from the Ct of gene of interest for each sample. The ΔΔCt was calculated by subtracting the ΔCt of the control sample from the ΔCt of each treated sample. Fold change was generated by using the equation 2^{-ΔΔCt}.

### Microscopy

The fluorescent dye Sytox® Green was used to investigate membrane permeabilization caused by PLNC8 αβ. This fluorophore can only cross damaged membranes and fluoresce upon binding to nucleic acids. Briefly, flaviviruses (LGTV or KUNV, 10⁵), suspended in DMEM cell culture medium, were either left untreated or exposed to L-PLNC8 αβ, D-PLNC8 αβ, or scrambled (S)-PLNC8 αβ (see Table 1) at a molar ratio of 1:1, or LL-37, for 2 h. The samples were then fixed with 4% PFA followed by addition of Sytox Green for 5 min, and images were captured with Olympus BX41 at 40× magnification. The images were processed and analysed using the software ImageJ.

Transmission electron microscopy (TEM) was used to visualize cells infected with LGTV and exposed to L-PLNC8 αβ or S-PLNC8 αβ. Briefly, LGTV (5×10⁴) were either left untreated or exposed to L-PLNC8 αβ or S-PLNC8 αβ for 1 h at a final concentration of 10 μM. The suspension was then added to Vero cells and incubated for 1 h, followed by washing with PBS and addition of fresh media. After 24 h of incubation, the cells were washed with PBS and fixed with 4% glutaraldehyde solution in 0.1 M phosphate buffer, pH 7.3. Specimens were washed in 0.1M phosphate buffer, postfixed in 2% osmium tetroxide in 0.1M phosphate buffer for 2 h and embedded into LX-112 (Ladd, Burlington, Vermont, USA). Ultrathin sections (approximately 50-60 nm) were cut by a Leica ultracut UCT/Leica EM UC 6 (Leica, Wien, Austria). Sections were contrasted with uranyl acetate followed by lead citrate and examined in a Hitachi HT 7700 (Tokyo, Japan). Digital images were taken by using a Veleta camera (Olympus Soft Imaging Solutions, GmbH, Münster, Germany).

Liposomes containing 5% 2-Oleoyl-1-palmitoyl-sn-glycero-3-phospho-rac-(1-glycerol) (POPG) and with or without cholesterol, were exposed to 1 μM of PLNC8 αβ for 2 min. Cryo-EM specimens were prepared by applying 3 μl liposome mixtures on Quantifoil grids 2/2 coated with a 2 nm carbon layer. Images where collected on a Talos Arctica 200 kV microscope (Thermo Fischer Scientific) using a Falcon3 direct electron detector in linear mode and at a magnification corresponding to a pixel size of 5 Å.

### Example 1

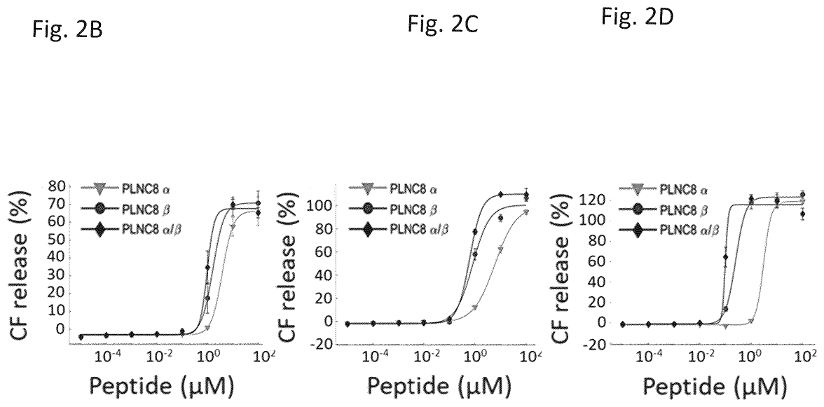

Our previous results showed that cholesterol-containing phospholipid bilayers are resistant to the membrane perturbing activity of PLNC8 αβ¹⁵. These results were confirmed herein by using Cryo-Electron Microscopy (Cryo-EM) that shows severe PLNC8 αβ-induced deformation of liposomes without cholesterol, compared to the resistant cholesterol-containing liposomes that were unaffected (FIG. 2A). FIG. 2A shows Cryo-EM images of liposomes containing 5% phosphatidylserine, without cholesterol (POPC:POPS 95:5; left-hand column) and with 30% cholesterol (POPC:POPS:Chol 65:5:30; right-hand column), respectively. The liposomes were either left untreated (Control; upper row) or exposed to L-PLNC8 αβ (lower row) at a final concentration of 1 μM for 2 min. Liposomes without cholesterol were deformed (black arrow heads) by the peptides and no longer associated with the carbon which lines the edges of the grid, indicating that they have lost their charge. Thus, a cholesterol content of 30% stabilized the phospholipid membranes against PLNC8 αβ. In summary, L-PLNC8 αβ is able to induce significant deformation of liposomes that did not contain cholesterol, while the cholesterol-containing liposomes were unaffected with a maintained round shape.

FIG. 2B shows that liposomes with the lipid composition POPC:POPS:Chol (70:0:30), mimicking a eukaryotic plasma membrane, is resilient towards PLNC8 αβ. FIG. 2C shows that by decreasing the amount of cholesterol and introducing a minimum amount of negative charge by using the lipid composition POPC:POPS:Chol (91:1:8), the liposome model becomes more susceptible to PLNC8 αβ. FIG. 2D shows that liposomes with even higher negative charge by using the lipid composition POPC:POPS:Chol (81:11:8), mimicking a eukaryotic ER membrane, are very susceptible towards PLNC8 αβ.

### Example 2

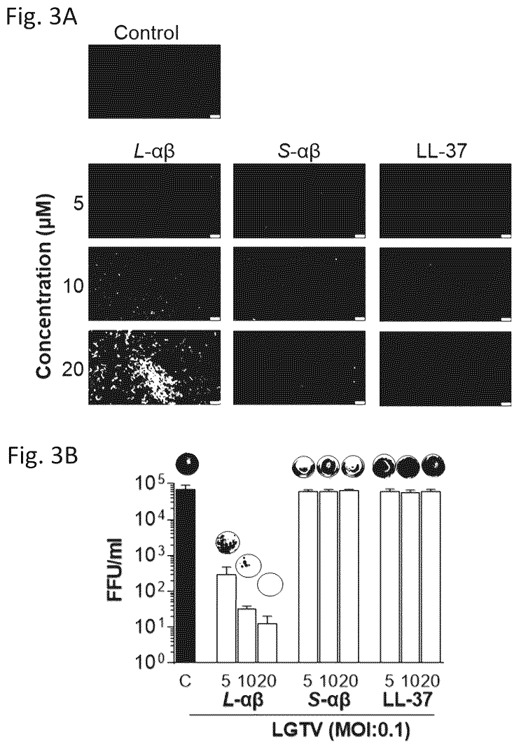

The antiviral activity of PLNC8 αβ was initially tested on the Flavivirus Langat (LGTV) that uses the rough ER for translation, assembly, and budding²⁶. The LGTV (1×10⁵), in DMEM, was either left untreated (Control) or exposed to L-PLNC8 αβ (1:1), scrambled S-PLNC8 αβ (1:1) or LL-37 at a final concentration of 5, 10 and 20 μM. The samples were fixed with 4% PFA after 2 h exposure, followed by addition of Sytox Green for 5 min. Images were captured with Olympus BX41 at 40× magnification. Scale bar is 200 μm. FIG. 3A shows that full-length L-PLNC8 αβ caused rapid permeabilization of the viral envelope, while neither the scrambled (S) variant of PLNC8 αβ (S-PLNC8 αβ; see Table 1) nor the human cathelicidin-derived peptide LL-37 (a known bactericidal peptide) caused permeabilization. Further, LGTV was incubated in cell culture media containing the indicated concentrations of L-PLNC8 αβ, S-PLNC8 αβ or LL-37 for 1 h at room temperature. The suspension was then added to Vero cells and incubated for 1 h. An overlay media was casted onto the cells and the plates were incubated for 3 days. Viral load was quantified by performing plaque assay. The indicated concentrations are in μM. Representative images of the virus plaques are presented above the bars in FIG. 3B. In said representative images, a lack of virus plaque staining indicates efficient elimination of virions in response to L-PLNC8 αβ. Quantification of the viral load after exposure of LGTV to the peptides for 1 h showed a dose-dependent decrease by L-PLNC8 αβ, resulting in >99% elimination of infective virions (FIG. 3B). The results indicate that the antiviral activity of PLNC8 αβ is specific since the effects were completely abolished when scrambling the sequences. In conclusion, the virus particles are rapidly permeabilized by L-PLNC8 αβ, forming large aggregates, and the viral load is decreased by >99.9%.

### Example 3

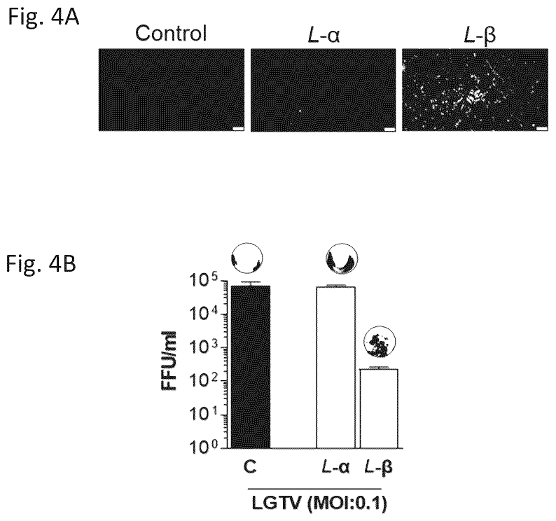

Next, LGTV (1×10⁵) was either left untreated (Control) or exposed to PLNC8 α or PLNC8 β at a final concentration of 20 μM. FIG. 4A shows permeabilization of LGTV by PLNC8 β, but not by L-PLNC8 α (Sytox Green staining, 2 h post-treatment). Scale bar is 200 μm. Further, LGTV was incubated in cell culture media containing 20 μM of either PLNC8 α or PLNC8 β for 1 h at room temperature, followed by infection of Vero cells for 1 h. Viral load was quantified by performing an immunofocus-based plaque assay. The results are shown in FIG. 4B. The virus particles are rapidly permeabilized by PLNC8 β, but not PLNC8 α, and decreased the viral load by >99%. Representative images of the virus plaques are presented above the bars, where visualization of single virus plaques in response to L-PLNC8 β indicates a substantial reduction of the viral load. However, optimal antiviral activity was achieved when both peptides were present, as shown in FIG. 3A and FIG. 3B. L-PLNC8 αβ significantly lowered the required concentration to efficiently eliminate infective virions compared to PLNC8 β alone, thus highlighting the importance of PLNC8 α.

### Example 4

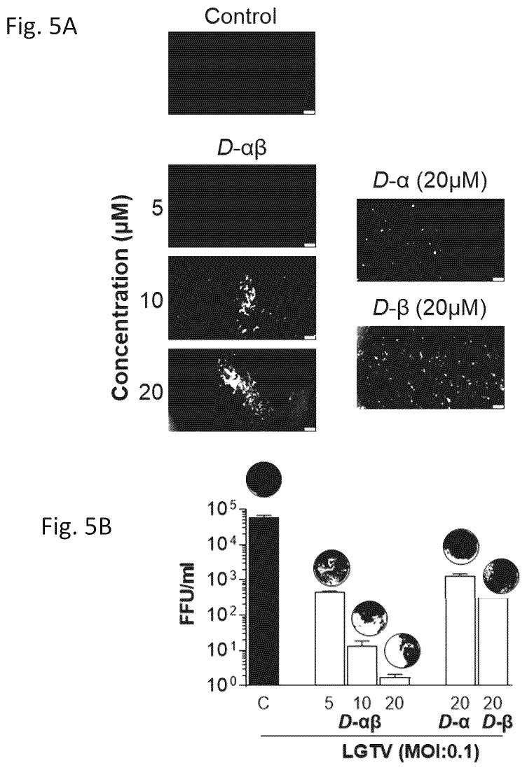

The findings described in Examples 2 and 3 encouraged us to determine if the antiviral activity of PLNC8 αβ was mediated through binding to a receptor on the surface of virions, which was investigated by using the D-enantiomers of the peptides that should not be able to bind to a protein target. Interestingly, the D-enantiomers of PLNC8 αβ showed potent antiviral properties, resulting in rapid permeabilization (FIG. 5A) and efficient elimination of LGTV (FIG. 5B).

More particularly, flavivirus LGTV (1×10⁵) was exposed to D-PLNC8 α, D-PLNC8 β at a final concentration of 20 μM or D-PLNC8 αβ (1:1) at a final concentration of 5, 10 and 20 μM. FIG. 5A shows that LGTV particles were permeabilized by D-PLNC8 β and D-PLNC8 αβ after 2 h of exposure, while D-PLNC8 α was not as efficient. Scale bar is 200 μm. Further, LGTV was incubated in cell culture media containing the indicated concentrations of D-PLNC8 αβ, either alone or together at a molar ratio of 1:1 for 1 h at room temperature. The suspension was then used to infect Vero cells 1 h followed by quantification of the viral load using immunofocus assay. The indicated concentrations in FIG. 5B are in μM. Representative images of the virus plaques are also presented above the bars in FIG. 5B, where reduced virus plaque staining indicates efficient elimination of virions in response to D-PLNC8 αβ. In conclusion, the D-enantiomer of PLNC8 αβ is efficient against the enveloped flavivirus LGTV. The virus particles are rapidly permeabilized by D-PLNC8 αβ, forming large aggregates, and decreased the viral load by >99.9%. D-PLNC8 β was more efficient than D-PLNC8 α at permeabilizing LGTV.

The results indicate that PLNC8 αβ does not bind to a specific receptor on the flavivirus Langat, suggesting that the binding is initiated by electrostatic interactions between the cationic peptides and anionic structures on virions, e.g., phospholipids in the viral envelope.

### Example 5

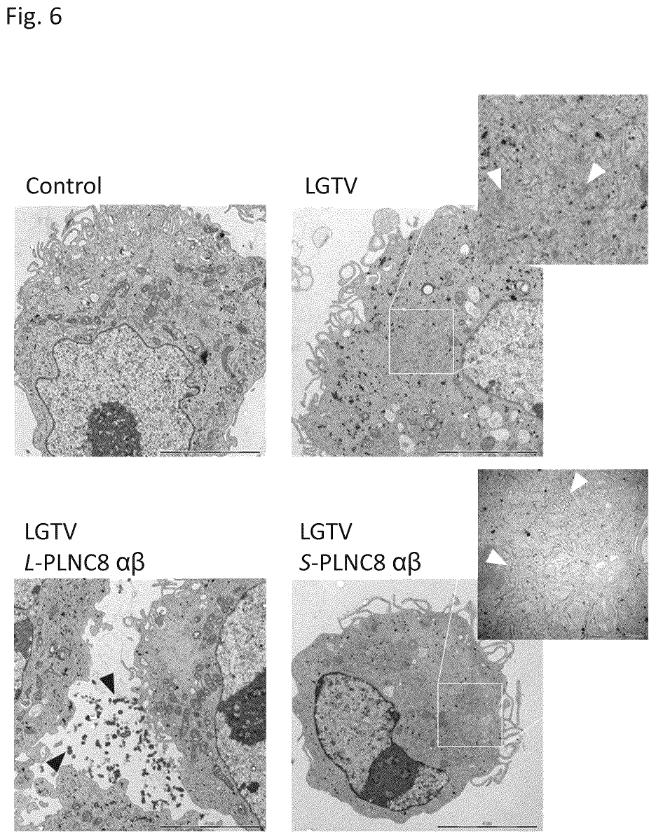

Furthermore, the antiviral effect of L-PLNC8 αβ was visualized under transmission electron microscopy (TEM) by the lack of intracellular virus-induced single-membrane vesicles that were clearly visible in LGTV-infected cells and in samples pre-treated with scrambled PLNC8 αβ (FIG. 6). More particularly, LGTV (1×10⁵) were pre-treated with 10 μM of L-PLNC8 αβ or S-PLNC8 αβ for 1 h prior to infection of Vero cells for 1 h, followed by washing addition of fresh media. After 24 h of incubation, the cells were fixed with 4% glutaraldehyde solution, processed, and visualized using TEM. Black arrow heads point towards formation of large aggregates with L-PLNC8 αβ and white arrow heads point on the presence of virus-induced single-membrane vesicles within the lumen of rough ER.

Interestingly, exposure of LGTV to L-PLNC8 αβ resulted in the formation of large, electron-dense aggregates, which may be a consequence of rapid and efficient permeabilization of virions, as these large aggregates were also visible under fluorescence microscopy in response to L-PLNC8 αβ, but not scrambled peptides (FIG. 3).

### Example 6

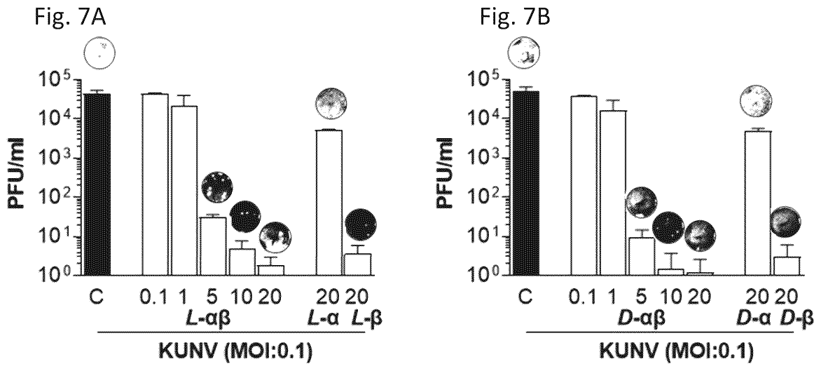

The antiviral activity of PLNC8 αβ on enveloped viruses was verified by using the virus Kunjin (KUNV), a flavivirus that is closely related to LGTV. Like LGTV, it derives its lipid envelope from the ER. KUNV (1×10⁵), suspended in DMEM, was exposed to L-enantiomer (FIG. 7A) or D-enantiomer (FIG. 7B) of PLNC8 α, PLNC8 β at a final concentration of 20 μM or PLNC8 αβ (molar ratio 1:1) at a final concentration of 0.1, 1, 5, 10 and 20 μM for 1 h at room temperature. The suspension was then added to Vero cells and incubated for 1 h, followed by quantification of the viral load by performing plaque assay. The indicated concentrations are in μM. Representative images of the virus plaques are presented above the bars. These images show enhanced staining of cells indicating cell survival, as a result of efficient elimination of virions in response to L-PLNC8 αβ and D-PLNC8 αβ, respectively, in a dose-dependent manner. The virus particles are rapidly permeabilized by both the L-enantiomer and the D-enantiomer of PLNC8 αβ, forming large aggregates (data not shown), and the viral load is decreased by >99.9%. Similarly, it was found that PLNC8 β alone was more efficient than PLNC8 α at eliminating KUNV (FIG. 7).

### Example 7

Furthermore, PLNC8 αβ efficiently eliminated KUNV even at low peptide concentrations and regardless of the initial viral load. KUNV, at a multiplicity of infection (MOI) of 0.1, 0.01, and 0.001 were exposed to increasing concentrations of either L-PLNC8 αβ or D-PLNC8 αβ (1:1) for 1 h, followed by infections of Vero cells and quantification of the viral load. A final peptide concentration of 0.1 μM and 1 μM reduced the viral load by 80% and 95%, respectively, while concentrations of ≥10 μM completely eliminates all virions (FIG. 8). Thus, it has been shown that both the L-enantiomer and the D-enantiomer of PLNC8 αβ can reduce the viral load even at low concentrations (0.01 μM).

### Example 8

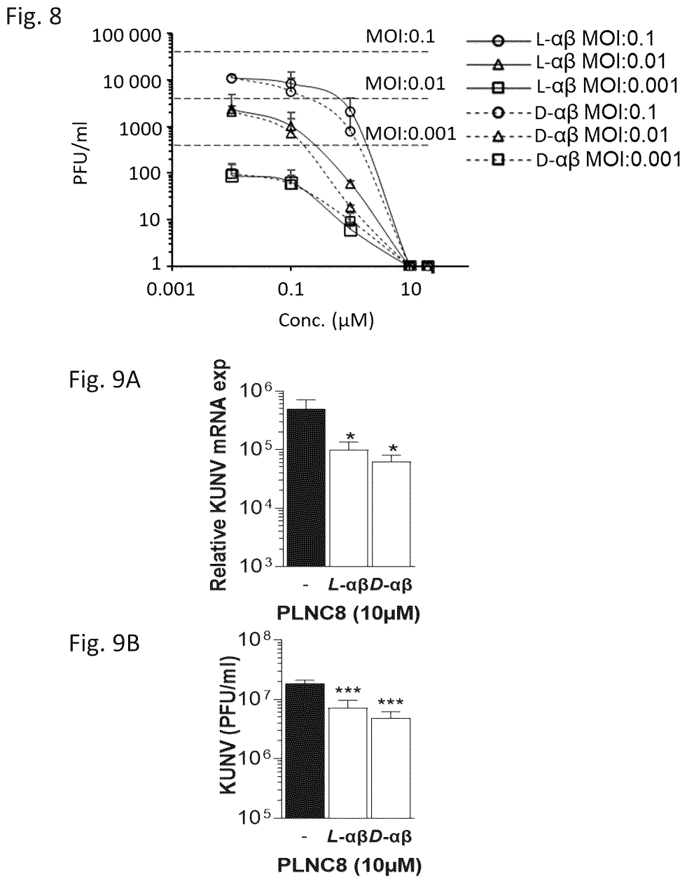

The following experiments were designed to investigate the antiviral activity of PLNC8 αβ on cells infected with KUNV, to determine if the peptides can affect virus replication, intracellular viral load, and accumulation of extracellular virions. Human lung carcinoma cells (A549) were infected with KUNV for 1 h, followed by removal of the cell culture medium and addition of fresh media (DMEM supplemented with 2.5% FBS) containing L- or D-PLNC8 αβ at a final concentration of 10 μM. The cell culture medium was collected after 48 h, and the cells were harvested. Viral replication was quantified by RT-PCR. A single dose of L-PLNC8 αβ or D-PLNC8 αβ reduced KUNV mRNA levels by 78% and 87%, respectively (FIG. 9A). Interestingly, both enantiomers were equally efficient at counteracting virus-induced cytotoxicity, as the number of viable cells was enhanced compared to the cell viability in infected and untreated cells (Control; white bar). PLNC8 αβ reduced the extracellular virion load (FIG. 9B) by 70-80%. Extracellular virions were quantified in the cell culture media. The substantial reduction in viral mRNA levels and intracellular virions may be a consequence of efficient elimination of extracellular virions, i.e., prevention of virus dissemination between cells. However, whether the peptides can cross the plasma membrane and interfere with virus replication and/or target intracellular viruses is currently under investigation. Thus, it has been shown that PLNC8 αβ targets mature extracellular viruses and causes a substantial reduction of extracellular virions.

Our findings of the antiviral properties of PLNC8 αβ prompted us to determine its activity against other enveloped viruses, e.g., SARS-CoV-2.

### Example 9

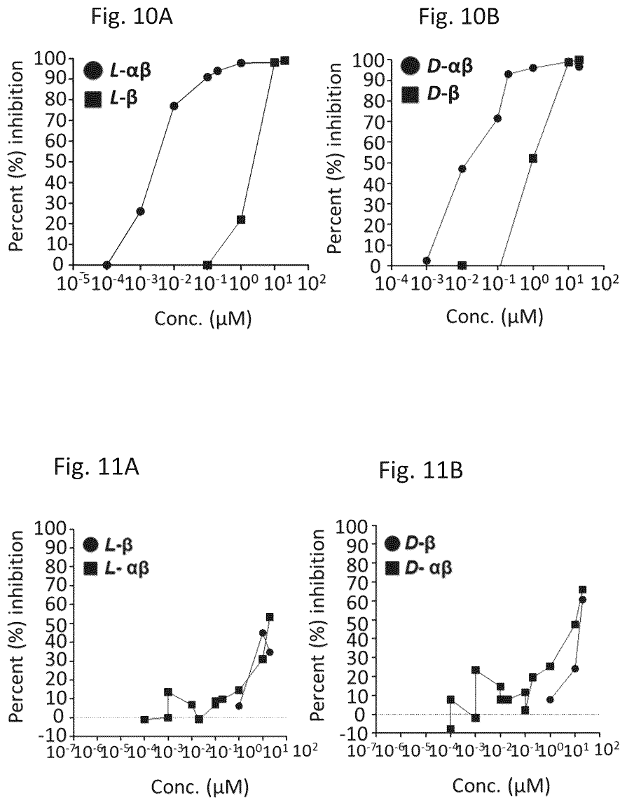

SARS-CoV-2 virus (1×10³) were either left untreated (data not shown) or exposed to L-enantiomer (FIG. 10A) or D-enantiomer (FIG. 10B) of PLNC8 β or PLNC8 αβ (1:1) using the indicated concentrations for 1 h at 37° C. The suspension was then used to infect Vero cells and the viral load was quantified by performing plaque assay. Both enantiomers of PLNC8 αβ caused a substantial reduction of the viral load in a dose-dependent manner. We have shown that both enantiomers of PLNC8 αβ are remarkably efficient against SARS-CoV-2, causing a substantial reduction of the viral load in a dose-dependent manner (FIG. 10). A 50% reduction of PFU with the L-form of PLNC8 αβ was achieved at 0.01 μM, while L-PLNC8 β alone required 0.9-1 μM with the used SARS-CoV-2 virus concentration (FIG. 10A). The D-form of PLNC8 αβ was more effective against SARS-CoV-2 and a 50% reduction was achieved at a final concentration of 0.005 μM, while D-PLNC8 β alone required 1.4 μM (FIG. 10B).

The lipid envelope of flaviviruses is derived from the ER, while coronaviruses exploit the vesicular-tubular trafficking system between the ER and Golgi apparatus, i.e., ER-Golgi intermediate compartments (ERGIC)²⁷⁻²⁹. The membrane characteristics of the ER and Golgi apparatus are similar, in that both have exposed anionic lipids in their outer leaflets and contain low levels of cholesterol, compared to the plasma membrane^{17,18}. The hypothesis of this study is based on knowledge describing these membrane characteristics and the apparent membrane activity of PLNC8 αβ, in addition to our previous results showing that PLNC8 αβ displays no cytotoxic effects. The hypothesis was tested by investigating the antiviral activity of PLNC8 αβ against enveloped viruses that gain their lipid wall from the plasma membrane, e.g., influenza A virus and HIV-1.

### Example 10

Influenza A virus (1×10³) were pre-treated with L-enantiomer (FIG. 11A) or D-enantiomer (FIG. 11B) of PLNC8 β or PLNC8 αβ (1:1) for 1 h at 37° C. The suspension was used to infect Vero cells followed by quantification of the viral load. L-PLNC8 αβ caused a 50% reduction of influenza A PFU at 20 μM (FIG. 11A), while 10 μM of D-PLNC8 αβ was sufficient to cause the same reduction in PFU (FIG. 11B). Higher concentrations of PLNC8 β alone were required to suppress influenza A, however the D-form was more effective than the L-form. Influenza A was suppressed by 50% at 19 μM of the D-form and at >20 μM of the L-form of PLNC8 β, respectively.

In conclusion, it has been shown that elimination of influenza A requires 100- to 1000-fold higher peptide concentrations to achieve an effect comparable to the antiviral effect of the peptides on flaviviruses and SARS-CoV-2.

### Example 11

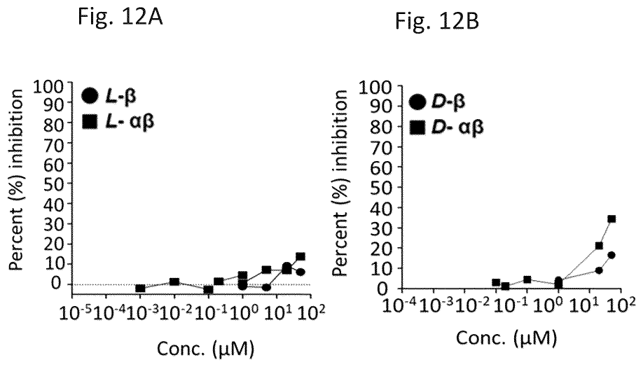

The HIV-1 (1×10³) were treated with L-enantiomer (FIG. 12A) or D-enantiomer (FIG. 12B) of PLNC8 β or PLNC8 αβ (1:1) for 1 h at 37° C. The suspensions were then used to infect isolated primary PBMC followed by quantification of the viral load. HIV-1 was shown to be less susceptible to PLNC8 αβ as a final concentration of 50 μM caused approximately 20% inhibition with the D-enantiomer. However, 50% reduction concentrations were not possible to calculate with the used HIV-1 virus titer (TCID50/0.1 ml) (FIG. 12A-B). In conclusion, it has been shown that elimination of HIV-1 requires 10³- to 10⁴-fold higher peptide concentrations to achieve an effect comparable to flaviviruses and SARS-CoV-2.

### Example 12

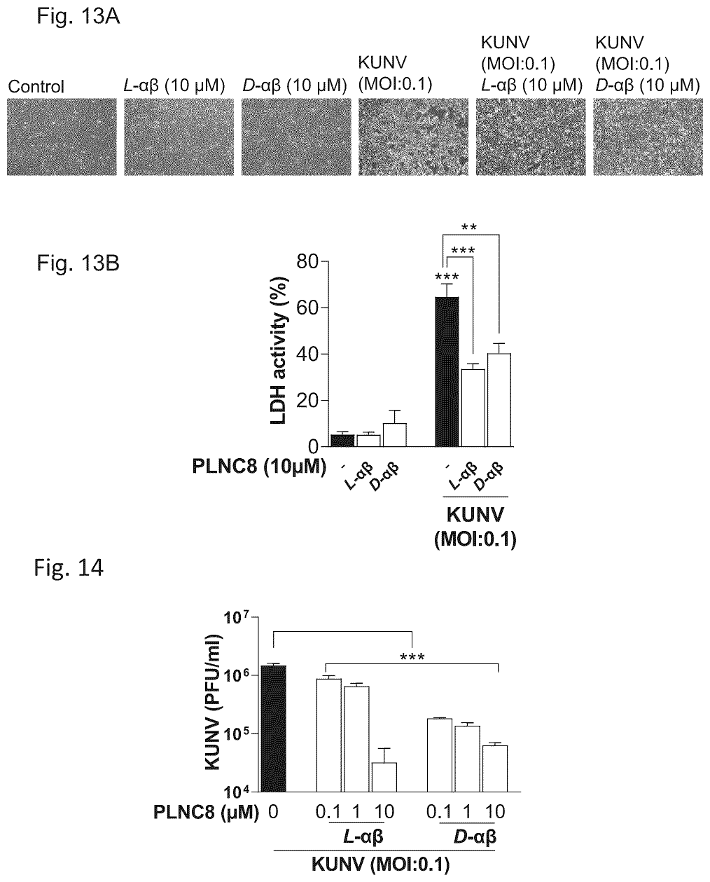

Moreover, host-cell inflammatory responses were analyzed after infection of human cells with KUNV, with or without the presence of the PLNC8 αβ. The results in Table 2 below show that gene expression of 12 genes (tlr3, tlr9, nod1, nod2, rank1, c-fos, c-jun, il-1β, il-6, tnf-α, cxcl8, and ccl-5) are slightly altered by the peptides L-αβ and D-αβ, respectively. Human lung carcinoma cells (A549) were infected with KUNV for 1 h, followed by treatment with 10 μM of L- or D-PLNC8 αβ for 48 h. Gene expression of inflammatory markers was analyzed with RT-PCR. KUNV induced gene expression of all analyzed inflammatory markers, except nod1 and rank1, and the presence of the peptides altered these effects by promoting cell survival. Furthermore, microscopical analysis showed that both enantiomers of PLNC8 αβ counteracted KUNV-induced cytotoxicity (FIG. 13A). Quantitative cytotoxicity data are presented in FIG. 13B.

TABLE 2
Gene expression analysis in response to KUNV and PLNC8 αβ. Human
lung carcinoma cells (A549) were infected with KUNV for 1 h, followed
by treatment with 10 μM of L-PLNC8 αβ (L-αβ) or D-
PLNC8 αβ (D-αβ) for 48 h. Gene expression was analyzed
with RT-PCR. The data (n = 3) are presented as mean with SD.

| | | | KUNV | | |
|---|---|---|---|---|---|
| | | | (MOI: 0.1) | | |
| Gene | L-αβ | D-αβ | — | L-αβ | D-αβ |
| tlr3 | 0.9 ± 0.5 | 0.4 ± 0.1 | 9.5 ± 3.3 | 26.7 ± 4.7 | 18.3 ± 5.9 |
| tlr9 | 2.9 ± 1.8 | 3.1 ± 2.1 | 11.3 ± 6.6 | 0.9 ± 0.6 | 3.6 ± 1.7 |
| nod1 | 0.9 ± 0.6 | 1.0 ± 0.7 | 0.7 ± 0.2 | 1.5 ± 0.4 | 2.1 ± 0.5 |
| nod2 | 0.9 ± 0.3 | 0.2 ± 0.1 | 88.1 ± 37.5 | 27.7 ± 8.0 | 22.8 ± 6.7 |
| rankl | 1.0 ± 0.6 | 1.9 ± 0.9 | 0.7 ± 0.2 | 4.2 ± 2.0 | 2.1 ± 1.1 |
| c-fos | 0.6 ± 0.4 | 0.9 ± 0.5 | 19.9 ± 8.6 | 4.9 ± 3.2 | 0.9 ± 0.4 |
| c-jun | 0.7 ± 0.3 | 1.3 ± 0.4 | 9.5 ± 2.9 | 3.4 ± 1.6 | 2.5 ± 1.0 |
| il-1β | 1.9 ± 1.0 | 1.7 ± 0.8 | 10.2 ± 3.6 | 5.1 ± 3.2 | 1.7 ± 0.7 |
| il-6 | 1.3 ± 1.7 | 2.8 ± 1.0 | 120.4 ± 42.2 | 556.9 ± 295.1 | 276.1 ± 165.6 |
| tnf-α | 0.8 ± 0.6 | 1.5 ± 2.9 | 90.4 ± 73.4 | 36.7 ± 30.2 | 6.5 ± 4.5 |
| cxcl-8 | 0.8 ± 0.6 | 0.9 ± 0.6 | 68.5 ± 11.3 | 24.9 ± 17.1 | 7.3 ± 8.1 |
| ccl-5 | 0.9 ± 0.2 | 0.9 ± 0.2 | 7.1 ± 3.2 | 25.5 ± 11.9 | 31.3 ± 8.8 |

### Example 13

In the following experimental setup, three doses of PLNC8 αβ were administered to KUNV-infected cells, once every 24 h, at three different concentrations (0.1, 1, and 10 μM). The viral load was quantified after 72 h and showed that a final concentration of L-PLNC8 αβ at 0.1, 1, and 10 μM caused 40%, 60%, and >90% inhibition of extracellular virions, respectively (FIG. 14). The D-form of PLNC8 αβ was markedly more potent at low concentrations (0.1 μM and 1 μM) than the L-form, causing 80-96% reduction of the number of infective virions.

### Example 14

In vivo experiments are performed as follows. Development of formulas with PLNC8 αβ will be mainly focused on topical applications, which is a non-invasive and effective approach. The administration route will be intranasal to cover the largest area possible of epithelial surface. Transgenic hACE2 mice (Mus musculus), hamsters (Mesocricetus auratus), and/or ferrets (Mustela putorius) will be used as animal models, with intranasal infection with SARS-CoV-2, that have been shown to develop symptoms similar to humans with COVID-19. Animal experiments will be performed under the strict regulation of the Ethics Committee for Animal Experimentation, with all the appropriate ethical permissions. The antiviral activity of novel peptides will be tested after intranasal administration, for preventive (administration before infection with SARS-CoV-2) and treatment purpose (administration after infection with SARS-CoV-2). Changes in health status including body temperature, weight, movement and posture, possible skin changes, appetite, bowel function, and breathing will be documented daily. Blood samples will be collected during the experiment to determine effects on the immune system, inflammatory responses, hematological and chemical changes, and virus titer using standardized laboratory techniques (flow cytometry, multiplex analysis, ELISA, RT-PCR, immunohistochemistry, plaque assay). Furthermore, nasal and lung lavage fluid will also be collected. These results will provide proof of concept for a therapeutic effect by preventing and/or treating virus infections with the antiviral peptide PLNC8 αβ.

### EXAMPLE CLAUSES

A: A pharmaceutical composition for use in the prevention and/or treatment of a viral infection and/or a disorder associated with a viral infection, caused by an enveloped virus, wherein the pharmaceutical composition comprises: (a) peptide having at least 90%, such as 95%, 96%, 97%, 98%, or 99%, sequence identity to an amino acid sequence according to SEQ ID NO: 1; or (b) a peptide having at least 90%, such as 95%, 96%, 97%, 98%, or 99%, sequence identity to an amino acid sequence according to SEQ ID NO:2, and a peptide having at least 90%, such as 95%, 96%, 97%, 98%, or 99%, sequence identity to an amino acid sequence according to SEQ ID NO: 1.
B: The pharmaceutical composition for use according to paragraph A, wherein at least one amino acid residue of the peptide according to (a) or at least one amino acid residue of any one or both of the peptides according to (b), is a D-amino acid residue.
C: The pharmaceutical composition for use according to any of paragraphs A-B, wherein the pharmaceutical composition comprises from about 1 nM to about 1000 μM, such as from about 10 nM to about 100 μM, of the peptide according to (a), or of each of the peptides according to (b).
D: The pharmaceutical composition for use according to any of paragraphs A, B, and/or C, wherein the pharmaceutical composition comprises the peptides according to (b) at a molar ratio of from about 1:1 to about 20:1, such as from about 1:1 to about 10:1, such as about 1:1.
E: The pharmaceutical composition for use according to any of paragraphs A, B, C, and/or D, wherein the viral infection is caused by an enveloped virus selected from the group consisting of the following virus families: Coronaviridae, Flaviviridae, Herpesviridae, Orthomyxoviridae, Retroviridae, Paramyxoviridae, Filoviridae, Pneumoviridae, Arteriviridae, Asfarviridae, Bunyaviridae, Hepadnaviridae, Poxviridae, Togaviridae and Rhabdoviridae.
F: The pharmaceutical composition for use according to any of paragraphs A, B, C, D, and/or E, wherein the viral infection is caused by an enveloped virus, whose envelope is obtained from the endoplasmic reticulum, the golgi apparatus or the nuclear envelope of the infected cell, such as an enveloped virus selected from the group consisting of the following virus families: Coronaviridae, Flaviviridae, Herpesviridae, Arteriviridae, Asfarviridae, Bunyaviridae, Hepadnaviridae and Poxviridae, optionally wherein the viral infection is caused by Severe Acute Respiratory Syndrome Coronavirus (SARS-CoV), Severe Acute Respiratory Coronavirus 2 (SARS-CoV-2), Middle East Respiratory Syndrome Coronavirus (MERS-CoV), Herpes Simplex Virus 1 (HSV-1), Herpes Simplex Virus 2 (HSV-2), Epstein-Barr Virus (EBV), Human Cytomegalovirus (HCMV), Varicella-Zoster Virus (VZV), Dengue Virus (DENV), Zika Virus (ZIKV), West Nile Virus (WNV), Langat Virus (LGTV) or Tick-borne Encephalitis Virus (TBEV), optionally wherein the West Nile Virus (WNV) is of the subtype Kunjin virus (KUNV).
G: The pharmaceutical composition for use according to any of paragraphs A, B, C, D, E, and/or F, wherein the viral infection is a respiratory tract infection or a mucus layer infection.
H: The pharmaceutical composition for use according to any of paragraphs A, B, C, D, E, F, and/or G, wherein the composition is administered locally to the site of infection, or close to the site of infection, such as topically.
I: The pharmaceutical composition for use according to any of paragraphs A, B, C, D, E, F, G, and/or H, wherein the composition is formulated as a powder, a solution, a cream, a gel, an ointment, or is formulated in immobilized form as a coating on a medical device; optionally wherein the solution is an aerosol and/or in the form of a nasal spray or mouth spray; optionally wherein the medical device is a face mask, an air filter, a nasal cannula device or an endotracheal tube.
J: The pharmaceutical composition for use according to any of paragraphs A, B, C, D, E, F, G, H, and/or I, wherein the pharmaceutical composition is formulated for administration as a single dose or multiple doses, such as two, three, four, or five doses per day, for 1-20 days.
K: The pharmaceutical composition for use according to any of paragraphs A, B, C, D, E, F, G, H, I, and/or J, wherein the disorder associated with a viral infection is selected from the group consisting of virus-induced inflammation, virus-induced cell death, virus-induced tissue destruction, and combinations thereof, optionally wherein the virus-induced tissue destruction is selected from the group consisting of damage of mucosal surfaces, pulmonary fibrosis, organ dysfunction, and combinations thereof.
L: A method treating a subject comprising administering to a subject in need of such a treatment a pharmaceutical composition according to any of paragraphs A, B, C, D, E, F, G, H, I, J, and/or K.

While the example clauses described above are described with respect to one particular implementation, it should be understood that, in the context of this document, the content of the example clauses can also be implemented via a method, device, system, and/or other implementations. Additionally, any of the example clauses A-L may be implemented alone or in combination with any other one or more of the example clauses.

### REFERENCES

ADDIN EN.REFLIST 1 Rouse, B. T. & Schrawat, S. Immunity and immunopathology to viruses: what decides the outcome? Nat Rev Immunol 10, 514-526. doi:10.1038/nri2802 (2010).
2 Hyodo, K. & Okuno, T. Pathogenesis mediated by proviral host factors involved in translation and replication of plant positive-strand RNA viruses. Curr Opin Virol 17, 11-18, doi:10.1016/j.coviro.2015.11.004 (2016).
3 Lou, Z., Sun, Y. & Rao, Z. Current progress in antiviral strategies. Trends Pharmacol Sci 35, 86-102, doi:10.1016/j.tips.2013.11.006 (2014).
4 Rome, B. N. & Avorn, J. Drug Evaluation during the Covid-19 Pandemic. N Engl J Med 382, 2282-2284, doi:10.1056/NEJMp2009457 (2020).
5 Watkins, J. Preventing a covid-19 pandemic. BMJ 368, m810, doi:10.1136/bmj.m810 (2020).
6 De Clereq, E. & Li, G. Approved Antiviral Drugs over the Past 50 Years. Clin Microbiol Rev 29, 695-747, doi:10.1128/CMR.00102-15 (2016).
7 Li, G. & De Clereq, E. Therapeutic options for the 2019 novel coronavirus (2019-nCoV). Nature reviews. Drug discovery 19, 149-150, doi:10.1038/d41573-020-00016-0 (2020).
8 Zhao, H. et al. A broad-spectrum virus- and host-targeting peptide against respiratory viruses including influenza virus and SARS-CoV-2. Nat Commun 11, 4252, doi:10.1038/s41467-020-17986-9 (2020).
9 Wolf, M. C. et al. A broad-spectrum antiviral targeting entry of enveloped viruses. Proc Natl Acad Sci USA 107, 3157-3162, doi:10.1073/pnas.0909587107 (2010).
10 Elnagdy, S. & AlKhazindar, M. The Potential of Antimicrobial Peptides as an Antiviral Therapy against ACS COVID-19. ACS Pharmacol Transl Sci 3, 780-782, doi:10.1021/acsptsci.0c00059 (2020).
11 Easom, N. et al. Sixty-eight consecutive patients assessed for COVID-19 infection: Experience from a UK Regional infectious diseases Unit. Influenza Other Respir Viruses 14, 374-379, doi:10.1111/irv.12739 (2020).
12 Duployez, C. et al. Panton-Valentine Leukocidin-Secreting Staphylococcus aureus Pneumonia Complicating COVID-19. Emerg Infect Dis 26, 1939-1941, doi:10.3201/cid2608.201413 (2020).
13 Maldonado, A., Ruiz-Barba, J. L. & Jimenez-Diaz, R. Purification and genetic characterization of plantaricin NC8, a novel coculture-inducible two-peptide bacteriocin from Lactobacillus plantarum NC8. Applied and environmental microbiology 69, 383-389 (2003).
14 Maldonado, A., Ruiz-Barba, J. L. & Jimenez-Diaz, R. Production of plantaricin NC8 by Lactobacillus plantarum NC8 is induced in the presence of different types of gram-positive bacteria. Archives of microbiology 181, 8-16, doi:10.1007/s00203-003-0606-8 (2004).
15 Bengtsson, T. et al. Plantaricin NC8 alphabeta exerts potent antimicrobial activity against Staphylococcus spp. and enhances the effects of antibiotics. Sci Rep 10, 3580, doi:10.1038/s41598-020-60570-w (2020).
16 Bengtsson, T. et al. Dual action of bacteriocin PLNC8 alphabeta through inhibition of Porphyromonas gingivalis infection and promotion of cell proliferation. Pathogens and disease 75, doi:10.1093/femspd/ftx064 (2017).
17 Casares, D., Escriba, P. V. & Rossello, C. A. Membrane Lipid Composition: Effect on Membrane and Organelle Structure, Function and Compartmentalization and Therapeutic Avenues. International journal of molecular sciences 20, doi:10.3390/ijms20092167 (2019).
18 van Meer, G., Voelker, D. R. & Feigenson, G. W. Membrane lipids: where they are and how 18 they behave. Nature reviews. Molecular cell biology 9, 112-124, doi: 10.1038/nrm2330 (2008).
19 Bangham, A. D., Standish, M. M. & Watkins, J. C. Diffusion of univalent ions across the lamellae of swollen phospholipids. Journal of molecular biology 13, 238-252, doi:10.1016/s0022-2836 (65) 80093-6 (1965).
20 Byrnes, J. R. et al. Competitive SARS-CoV-2 Serology Reveals Most Antibodies Targeting the Spike Receptor-Binding Domain Compete for ACE2 Binding. mSphere 5, doi:10.1128/mSphere.00802-20 (2020).
21 Haveri, A. et al. Serological and molecular findings during SARS-CoV-2 infection: the first case study in Finland, January to February 2020. Euro Surveill 25, doi: 10.2807/1560-7917.ES.2020.25.11.2000266 (2020).
22 Yu, J. et al. DNA vaccine protection against SARS-CoV-2 in rhesus macaques. Science 369, 806-811, doi: 10.1126/science.abc6284 (2020).
23 Truelove, S. et al. A comparison of hemagglutination inhibition and neutralization assays for characterizing immunity to seasonal influenza A. Influenza Other Respir Viruses 10, 518-524, doi:10.1111/irv.12408 (2016).
24 Sundqvist, V. A. et al. Human immunodeficiency virus type 1 p24 production and antigenic variation in tissue culture of isolates with various growth characteristics. J Med Virol 29, 170-175, doi: 10.1002/jmv.1890290305 (1989).
25 Devito, C., Levi, M., Broliden, K. & Hinkula, J. Mapping of B-cell epitopes in rabbits immunised with various gag antigens for the production of HIV-1 gag capture ELISA reagents. J Immunol Methods 238, 69-80, doi:10.1016/s0022-1759 (00) 00141-1 (2000).
26 Gerold, G., Bruening, J., Weigel, B. & Pietschmann, T. Protein Interactions during the Flavivirus and Hepacivirus Life Cycle. Mol Cell Proteomics 16, S75-S91, doi:10.1074/mcp.R116.065649 (2017).
27 Brian, D. A. & Baric, R. S. Coronavirus genome structure and replication. Curr Top Microbiol Immunol 287, 1-30, doi:10.1007/3-540-26765-4_1 (2005).
28 Risco, C. et al. Endoplasmic reticulum-Golgi intermediate compartment membranes and vimentin filaments participate in vaccinia virus assembly. J Virol 76, 1839-1855, doi:10.1128/jvi.76.4.1839-1855.2002 (2002).
29 Mohan, J. & Wollert, T. Membrane remodeling by SARS-CoV-2-double-enveloped viral replication. Fac Rev 10, 17, doi:10.12703/r/10-17 (2021).

1. A method for preventing and/or treating a viral infection and/or a disorder associated with a viral infection, caused by an enveloped virus, comprising administering a pharmaceutical composition,
wherein the pharmaceutical composition comprises:
a) a peptide having at least 90%, such as 95%, 96%, 97%, 98%, or 99%, sequence identity to an amino acid sequence according to SEQ ID NO:1;
or
b) a peptide having at least 90%, such as 95%, 96%, 97%, 98%, or 99%, sequence identity to an amino acid sequence according to SEQ ID NO:2, and a peptide having at least 90%, such as 95%, 96%, 97%, 98%, or 99%, sequence identity to an amino acid sequence according to SEQ ID NO:1.

2. The method according to claim 1, wherein the pharmaceutical composition further comprises at least one amino acid residue of the peptide according to (a) or at least one amino acid residue of any one or both of the peptides according to (b), is a D-amino acid residue.

3. The method according to claim 1, wherein the pharmaceutical composition further comprises from about 1 nM to about 1000 μM, such as from about 10 nM to about 100 μM, of the peptide according to (a), or of each of the peptides according to (b).

4. The method according to claim 1, wherein the pharmaceutical composition further comprises the peptides according to (b) at a molar ratio of from about 1:1 to about 20:1, such as from about 1:1 to about 10:1, such as about 1:1.

5. The method according to claim 1, wherein the viral infection is caused by an enveloped virus selected from the group consisting of the following virus families: Coronaviridae, Flaviviridae, Herpesviridae, Orthomyxoviridae, Retroviridae, Paramyxoviridae, Filoviridae, Pneumoviridae, Arteriviridae, Asfarviridae, Bunyaviridae, Hepadnaviridae, Poxviridae, Togaviridae and Rhabdoviridae.

6. The method according to claim 1, wherein the viral infection is caused by an enveloped virus, whose envelope is obtained from the endoplasmic reticulum, the golgi apparatus or the nuclear envelope of the infected cell,
such as an enveloped virus selected from the group consisting of the following virus families: Coronaviridae, Flaviviridae, Herpesviridae, Arteriviridae, Asfarviridae, Bunyaviridae, Hepadnaviridae and Poxviridae,
optionally wherein the viral infection is caused by Severe Acute Respiratory Syndrome Coronavirus (SARS-CoV), Severe Acute Respiratory Coronavirus 2 (SARS-CoV-2), Middle East Respiratory Syndrome Coronavirus (MERS-CoV), Herpes Simplex Virus 1 (HSV-1), Herpes Simplex Virus 2 (HSV-2), Epstein-Barr Virus (EBV), Human Cytomegalovirus (HCMV), Varicella-Zoster Virus (VZV), Dengue Virus (DENV), Zika Virus (ZIKV), West Nile Virus (WNV), Langat Virus (LGTV) or Tick-borne Encephalitis Virus (TBEV), optionally wherein the West Nile Virus (WNV) is of the subtype Kunjin virus (KUNV).

7. The method according to claim 1, wherein the viral infection is a respiratory tract infection or a mucus layer infection.

8. The method according to claim 1, wherein the composition is administered locally to the site of infection, or close to the site of infection, such as topically.

9. The method according to claim 1, wherein the composition is formulated as a powder, a solution, a cream, a gel, an ointment, or is formulated in immobilized form as a coating on a medical device;
optionally wherein the solution is an aerosol and/or in the form of a nasal spray or mouth spray;
optionally wherein the medical device is a face mask, an air filter, a nasal cannula device or an endotracheal tube.

10. The method according to claim 1, wherein the pharmaceutical composition is formulated for administration as a single dose or multiple doses, such as two, three, four, or five doses per day, for 1-20 days.

11. The method according to claim 1, wherein the disorder associated with a viral infection is selected from the group consisting of virus-induced inflammation, virus-induced cell death, virus-induced tissue destruction, and combinations thereof, optionally wherein the virus-induced tissue destruction is selected from the group consisting of damage of mucosal surfaces, pulmonary fibrosis, organ dysfunction, and combinations thereof.
